(19) United States
(12) Patent Application Publication
(10) Pub. No.: US 2024/0164889 A1
(43) Pub. Date: May 23, 2024

(54) AORTIC PROSTHESIS WITH TUNNEL GRAFT AND EMBOLIC FILTER

(71) Applicant: Bolton Medical, Inc., Sunrise, FL (US)

(72) Inventor: Amanda Casanova, Sunrise, FL (US)

(21) Appl. No.: 18/420,205

(22) Filed: Jan. 23, 2024

Related U.S. Application Data
(63) Continuation of application No. PCT/US22/38295, filed on Jul. 26, 2022.
(60) Provisional application No. 63/225,789, filed on Jul. 26, 2021.

(51) Int. Cl. A61F 2/07; A61F 2/01; A61F 2/06

(52) U.S. Cl. CPC A61F 2/07; A61F 2/01; A61F 2002/067

(57) ABSTRACT

An aortic graft assembly includes a tubular component that defines a wall aperture having a proximal end that extends perpendicular to a major longitudinal axis of the tubular aortic component, and a tunnel graft connected to the wall of the tubular aortic component and extending from the wall aperture toward a proximal end of the tubular aortic component. A filter that captures emboli consequent to implantation of the aortic graft assembly spans at least on of an aperture in the wall of the tubular aortic component and the tunnel graft. The method for delivery of the aortic graft assembly includes delivering the aortic graft assembly through the wall aperture and into interfering relation with the tunnel graft, thereby rupturing the filter, while the filter continues to retain captured emboli.

### CROSS-REFERENCE TO RELATED APPLICATION

This is a Continuation of International Application PCT/US22/38295, filed on Jul. 26, 2022, which claims the benefit of U.S. Provisional Patent Application No. 63/225,789, filed Jul. 26, 2021, the entire contents of each prior applications are incorporated herein by reference.

### BACKGROUND OF THE INVENTION

Aortic aneurysms are life-threatening conditions. Surgical interventions used to treat aortic aneurysms include endovascular repair by transluminal placement of one or more endografts across the longitudinal extent of the lesion. The endograft is placed in the aorta with the intention of bridging the aneurysmal sac to exclude it from the high-pressure of aortic blood flow, which can permit remodeling of the aortic wall in and around the aneurysm site. In certain regions of the aorta accurate placement of the endograft is critical to maintain blood flow to vessels branching from the aorta to minimize compromised blood flow to organs. For example, currently, if aortic devices are placed within the aortic arch in a manner that offsets the aperture for the left carotid artery, the artery can be occluded, which can result in ischemia to the brain. Most surgical methods of treating aneurysms at or near the aortic arch generally involve sternotomy or thoracotomy and may require cardio-pulmonary bypass, often resulting in high morbidity rates.

Further, trauma associated with implantation of endografts, particularly in regions of diseased tissue, poses a risk of stroke that may be associated with emboli created during the surgical procedure of deployment.

Thus, there is a need to develop new and useful devices and methods of treating aortic aneurysms by endovascular methods.

### SUMMARY OF THE INVENTION

The present invention relates to vascular repair systems, delivery systems and methods of using the delivery systems and its components to treat aortic vascular damage, in particular, vascular damage associated with aortic disease, such as, aneurysms, penetrating atherosclerotic ulcers and dissection.

In an embodiment, the invention is an aortic graft assembly that includes a tubular aortic component having a proximal end and a distal end connected by a wall of the tubular aortic component, the wall defining a wall aperture that is between the proximal and distal ends, the wall aperture having a proximal end and a distal end. A tunnel graft is connected to the wall of the tubular aortic component and extends from the wall aperture toward the proximal end of the tubular aortic component. The tunnel graft has a proximal end and a distal end, and defines a tunnel graft lumen that extends between the distal end and the proximal end of the tunnel graft lumen, the distal end of the tunnel graft lumen being at the wall aperture of the tubular aortic component. A proximal stent supports proximal end of the tubular aortic component. A distal stent supports the distal end of the tubular aortic component. At least one filter spans at least one of the aperture and the tunnel graft.

In another embodiment, the invention is a method for implanting a prosthesis that includes delivering an aortic graft assembly that includes a tubular aortic component through an aorta to an aneurysm site of a patient, the tubular aortic component defining a tunnel lumen and having a proximal end and a distal end connected by a wall, the wall defining a wall aperture that is between the proximal end and the distal end, the wall aperture having a proximal end and a distal end, the aortic graft assembly further including a tunnel graft extending from the wall aperture and within the tunnel lumen of the tubular aortic component toward the proximal end of the tubular aortic component, wherein at least one filter spans at least one of the aperture and the tunnel graft. The wall aperture is aligned over at least one vessel ostium at the aneurysm site of the patient. The outer tube is retracted, thereby releasing the tubular aortic component from the distal and proximal clasps, thereby deploying the tubular aortic component at the aneurysm site in the patient.

The aortic assembly systems and methods of the invention include a filter that sequesters, traps, or otherwise captures emboli that form, or become dislodged during implantation of aortic prostheses and branch prostheses, such as at the aortic arch. During implantation of a tubular aortic component into, for example, an aortic arch of a patient, emboli (i.e. a blood clot, air bubble, piece of fatty deposit, or other object that can be carried in the bloodstream to lodge in a vessel and cause an embolism), are formed that can escape the surgical site and be transported to other parts of the patient's anatomy and thereby cause stroke and, possibly, death. The filter component of the aortic assembly system of the invention captures emboli, thereby preventing their transport away from the surgical site.

The aortic assembly systems and methods of the invention can be employed to treat aortic aneurysms, such as aortic aneurysms at, near or around the arch of the aorta, or branches from the abdominal aorta (e.g., celiac artery, superior mesenteric artery and renal arteries). The aortic assembly systems of the invention have a relatively large aperture tapered into a tunnel graft that provides the surgeon with a relatively large margin of error in placement of the system, facilitates cannulation and permits alignment of a single aperture for at least one blood vessel. Aortic assembly systems of the invention that include a tunnel graft having one aperture extending proximally with two openings permit for easy alignment in the aorta, particularly in regions of the aorta that branch to peripheral and major vessels. The size of the aperture allows blood to flow to target vessels during the procedure. The aortic graft assembly of the invention generally does not restrict blood flow acutely or chronically, in part, because of a relatively large diameter of the tunnel graft and the stent or stents supporting the tunnel graft.

The invention can be used to treat various aortic pathologies, including aortic aneurysms, penetrating atherosclerotic ulcers, dissections and, therefore, avoid complications and death consequent to life-threatening vascular conditions.

### BRIEF DESCRIPTION OF THE DRAWINGS

FIG. 1 is a perspective view of an embodiment of an aortic assembly system of the invention wherein a filter of the invention is not visible.

FIG. 2 is a side view of the embodiment shown in FIG. 1.

FIG. 3A is a cross-sectional view of the embodiment shown in FIGS. 1 and 2, showing a tunnel graft of the aortic assembly system of the invention, and a filter between two stents supporting the tunnel graft.

FIG. 3B is a detail of the embodiment shown in FIG. 3A, showing the tunnel graft and filter within the tunnel graft.

FIG. 4A is a view of another embodiment of an aortic assembly system of the invention wherein the tunnel graft is bifurcated into two tunnel grafts at a point proximal to the proximal end of the aperture.

FIG. 4B is a view of the embodiment of the aortic assembly system of the invention of FIG. 4A taken along line 4B-4B.

FIG. 5A is a view of yet another embodiment of an aortic assembly system of the invention wherein the tunnel graft is bifurcated by a wall dividing a single tunnel graft.

FIG. 5B is a view of the embodiment of the aortic assembly system of the invention of FIG. 5A taken along line 5B-5B.

FIG. 6 is a cross-sectional view of still another embodiment of the aortic assembly system of the invention, wherein a filter spans the aperture of the tubular aortic component of the aortic assembly system of the invention.

FIG. 7 is a cross-sectional view of an embodiment of the aortic assembly system of the invention, wherein the tunnel graft includes four stents, each of which is partitioned from the others by a filter of the aortic assembly system.

FIG. 8 is a cross-sectional view of an embodiment of the aortic assembly system of the invention following implantation at an aortic arch of a patient.

FIG. 9 is a cross-sectional view of the implanted aortic assembly system shown in FIG. 8, following at least partial implantation of a branch stent graft in the brachiocephalic trunk, through the aperture and into the tunnel graft of the aortic assembly system.

FIG. 10 is a cross-section al view of the implanted aortic assembly system shown in FIGS. 8 and 9, following at least partial implantation of a branch stent graft in the left common carotid artery, through the aperture and into the tunnel graft of the aortic assembly system of the invention.

### DETAILED DESCRIPTION OF THE INVENTION

The features and other details of the invention, either as steps of the invention or as combinations of parts of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

“Proximal” means, when reference is made to a delivery system or a component of a delivery system, such as an apex clasp and a nose cone, closest to the clinician using device. Likewise, “distal” means, when reference is made to a delivery system or a component of a delivery system, such as an apex clasp and a nose cone, away from the clinician using the device.

When reference is made to a prosthesis to be delivered, such as an aortic graft assembly, tubular aortic component, tunnel graft, branch graft and stent, the word “proximal” means that portion of the prosthesis or component of the prosthesis that is towards the heart of the patient and “distal” means that portion of the prosthesis or component of the prosthesis that is away from the heart of the patient. For clarity, the word “proximate” means close to as opposed to “proximal” or “distal.”

Aortic graft assemblies of the invention can be implanted, for example, by trans femoral access. Tubular branch components can be implanted, for example, by supra aortic vessel access (e.g., brachial artery), or by trans femoral or trans apical access.

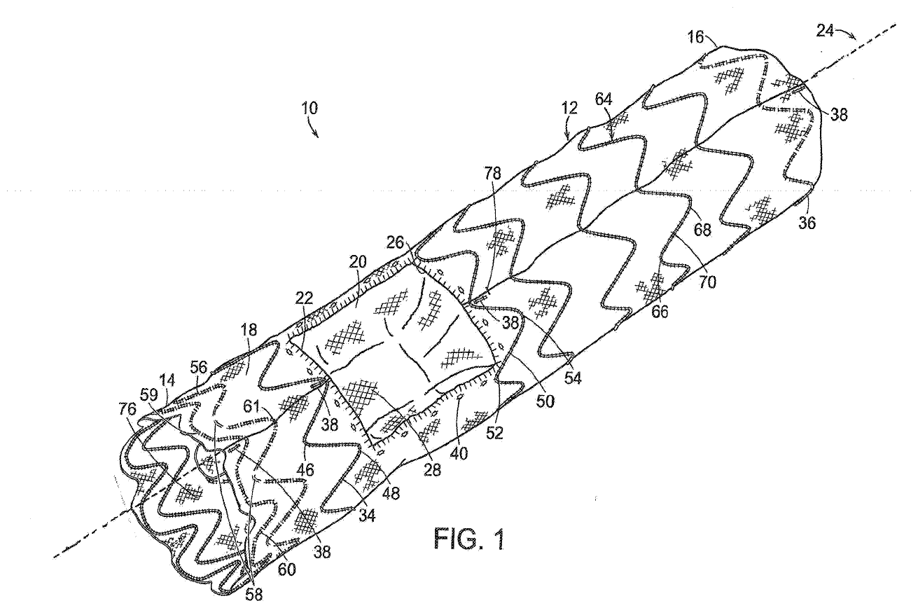

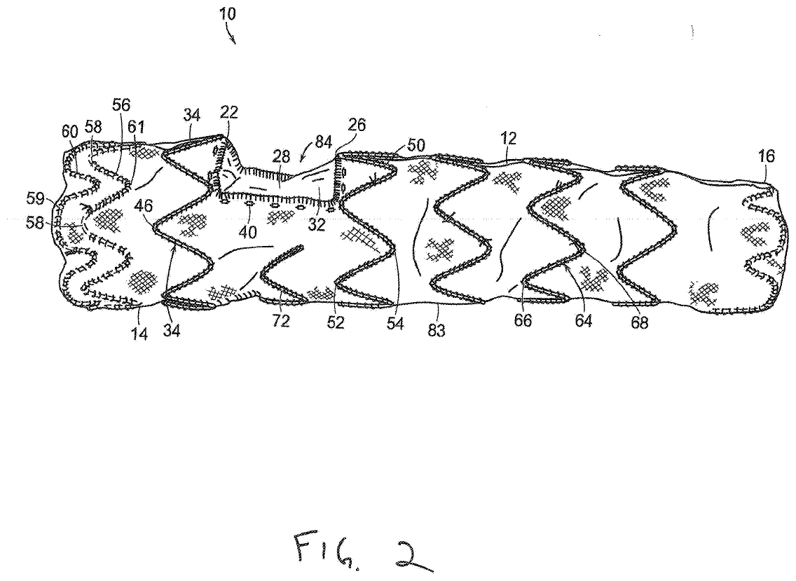

The invention is generally directed to an aortic graft assembly and a method for deploying the aortic graft assembly. The invention is also directed to methods of implanting at least one tubular branch graft into a patient and the aortic graft assembly. In one embodiment of the aortic graft assembly of the invention, represented in FIGS. 1 and 2, aortic graft assembly 10 includes tubular aortic component 12 having proximal end 14 and distal end 16 connected by wall 18. Wall 18 defines wall aperture 20 that is between proximal end 14 and distal end 16. Wall aperture 20 has proximal end 22 that extends perpendicular to a major longitudinal axis 24 of tubular aortic component 12 when viewed orthogonally to major longitudinal axis 24. Wall aperture 20 also defines distal end 26 of wall aperture 20.

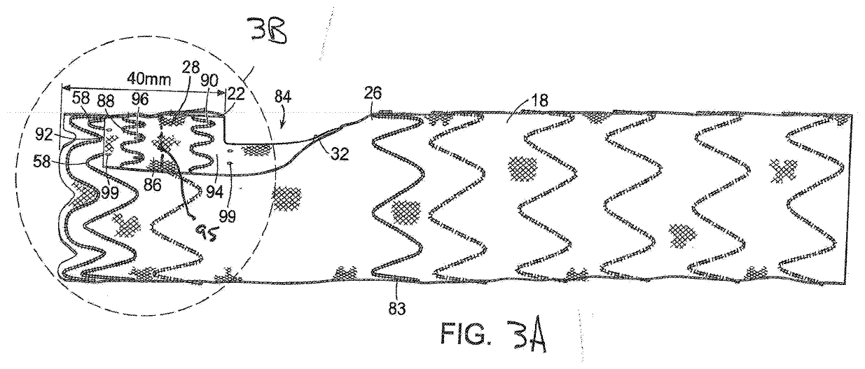

Tunnel graft 28, shown, for example, in FIGS. 3A and 3B, is connected to wall 18 of tubular aortic component 12 and extends from wall aperture 20 toward proximal end 14 of the tubular aortic component 12. Tunnel graft 28 includes proximal end 30 and distal end 32. Distal end 32 of tunnel graft 28 is at wall aperture 20 of tubular aortic component 12.

Referring back to FIGS. 1 and 2, proximal stent 34 supports proximal end 14 of tubular aortic component 12. Distal stent 36 supports distal end 16 of tubular aortic component 12. Similarly, distal stent 36 can be attached to an interior wall to tubular aortic component 12.

Optionally, radiopaque markers 38 are located along a line parallel to major longitudinal axis 24 of tubular aortic component 12. In one embodiment, radiopaque marker 38 is at a proximal apex of wall aperture distal stent 50 abutting wall aperture 20. Another radiopaque marker is at a distal apex 48 of proximal stent 34. Further, radiopaque marker 38 is at least one of proximal end 14 and distal end 16 of tubular aortic component 12. Also optionally, radiopaque markers 40 extend about the circumference of wall aperture 20 at tubular aortic component 12. Radiopaque markers 38, 40 can be made of any suitable material such as platinum, iridium, gold, etc. Examples of radiopaque markers are described in the U.S. Pat. Nos. 8,062,345 and 10,105,248, the entire teachings of which are incorporated herein by reference.

Proximal stent 34 in one embodiment, shown in FIGS. 1 and 2, includes proximal apices 46 and distal apices 48. In one embodiment, at least a portion of distal apices 48 abut proximal end 22 of wall aperture 20. Wall aperture distal stent 50 includes proximal apices 52 and distal apices 54, a portion of proximal apices 52 of wall aperture distal stent 50 abut distal end 26 of wall aperture 20. Clasping stent 56 at proximal end 14 of tubular aortic component 12 includes at least two exposed proximal apices 58 proximate to proximate end 14 of tubular aortic component 12. In one embodiment, clasping stent 56 is attached to an interior wall of tubular aortic component 12.

In one embodiment, crown stent 60 is located between clasping stent 56 and proximal end 14 of tubular aortic component 12. Crown stent 60 and clasping stent 56 can be nested, as shown in FIG. 1. Crown stent 60 and clasping stent 56 are attached to interior wall 76 of tubular aortic component 12.

In one embodiment, at least one stent 64 is located at tubular aortic component 12 between proximal stent 34 and distal stent 36. At least a portion of stents 64 include proximal apices 66 and distal apices 68 connected by struts 70. In one embodiment, at least one partial stent 72 is located at tubular aortic component 12 between stents 34, 50 abutting proximal end 22 and distal end 26 of wall aperture 20, respectively, as shown in FIG. 2.

Stents employed in the invention are constructed of a suitable material. In one embodiment, the stents employed by the invention include a suitable shape memory alloy, such as nitinol. Further description of suitable materials for construction of stents for use in the invention can be found in U.S. Pat. Nos. 7,763,063 and 8,062,345, the teachings of which are incorporated herein by reference in their entirety.

In one embodiment, tubular portion includes stents 88, 90 at each of a proximal 92 and distal end 94 of tubular portion 86, as shown in FIGS. 3A and 3B. Preferably, stents 88, 90 at proximal 92 and distal 94 ends of tubular portion 86 include proximal and distal apices connected by struts. Preferably, stent 88 at proximal end 92 of tubular portion 86 includes at least one barb 96 (FIGS. 3A and 3B).

Filter 95 spans tunnel graft 28 between stents 88 and 90, to avoid interference by stents 88 and 90. Filter 95 is sewn into tunnel graft 28. Filter 95, in one embodiment, has a mesh pattern and a porosity that will capture emboli, but allow passage of red blood cells. The diameter of the pores is suitable to for capture of emboli, while allowing passages of red blood cells. Examples of suitable ranges of pore sizes in the mesh include, for example, a range of between about 80 μm and about 90 μm, between a range of between about 90 μm and 100 μm, a range of between about 100 μm and 110 μm, and a range of between about 80 μm and about 110 μm. Filter 95 typically is biocompatible, since it will generally be left behind, in the body, following implantation of the aortic prosthesis system and any branch stent grafts into tunnel graft 28. Filter 95 also is sufficiently thin to allow perforation by a delivery device, such as a guidewire catheter, during implantation of branch stent grafts into tunnel 28, without allowing particles captured by filter 95 to be released into the patient's bloodstream. Filter 95 is also sufficiently stable to survive gamma sterilization. In one embodiment, the filter 95 is circumferentially sewn into the tunnel, and the caudal half of the sewn mesh is loosely sewn so that when the branch limb delivery system tip pushes on it, it breaks on the caudal portion. Examples of suitable materials of filter 95 include nylon, nitinol, and polyester, such as 50 polyester. During implantation of tubular aortic component 12 into, for example, an aortic arch of a patient, emboli (i.e. a blood clot, air bubble, piece of fatty deposit, or other object that can be carried in the bloodstream to lodge in a vessel and cause an embolism), can form, escape from the surgical site and be transported to other parts of the patient's anatomy, thereby increasing the likelihood of stroke and, possibly, death. Filter 95 captures emboli, thereby preventing their transport away from the surgical site, to decrease the likelihood of or prevent stroke, or other consequent injury.

In one embodiment, the arc length of proximal end 22 of wall aperture 20 is equal to or less than one-half the circumference of tubular aortic component 12. Examples of suitable arc lengths of proximal end 22 of wall aperture 20 include arc lengths equal to one member selected from the group consisting of about 6 mm, about 8 mm, about 10 mm, about 12 mm or about 14 mm. In one embodiment, a longitudinal length of wall aperture 20 is equal to or less than about 90 mm. In another embodiment, the longitudinal length of wall aperture 20 is equal to or greater than about 14 mm.

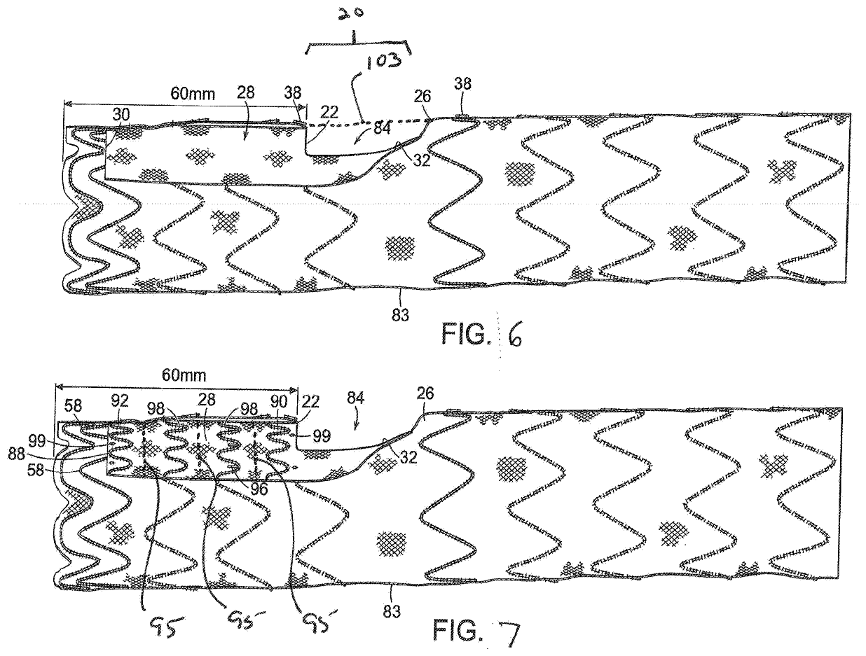

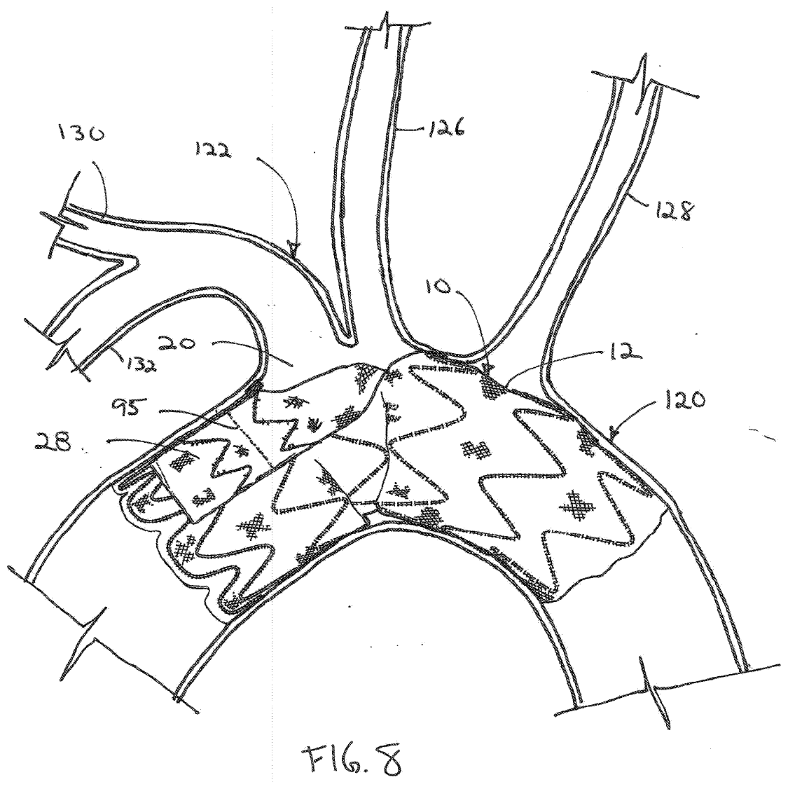

Referring to FIGS. 3A, 3B, 6, and 7, the distance between proximal end 22 of wall aperture 20 and proximal end 14 of tubular aortic component 12 can be in a range of between about 10 mm and about 80 mm. In a typical embodiment, the distance between proximal end 22 of wall aperture 20 and proximal end 14 of tubular aortic component 12 is one member selected from the group consisting of about 20 mm, about 40 mm, about 60 mm, about 80 mm or about 90 mm. In one embodiment, the distance between proximal end 22 of wall aperture 20 and proximal end 12 of tubular aortic component 12 is about 40 mm, as shown in FIG. 3A. In another embodiment, the distance between proximal end 22 of wall aperture 20 and proximal end 14 of tubular aortic component 12 is about 60 mm, as shown in FIGS. 6 and 7.

In one embodiment, shown in FIG. 1, retention component 78 is located at tubular aortic component 12 distal to wall aperture 20 and within tubular aortic component 12 (only external portion of retention component 78 is shown in FIG. 1). In one embodiment, retention component is a suture loop. In another embodiment, retention component 78 is at least one of a magnet or a stent apex. In still another embodiment, retention component 78 is radiopaque. In one embodiment, retention component 78 is at a proximal apex 52 of stent 50 abutting distal end 26 of wall aperture 20.

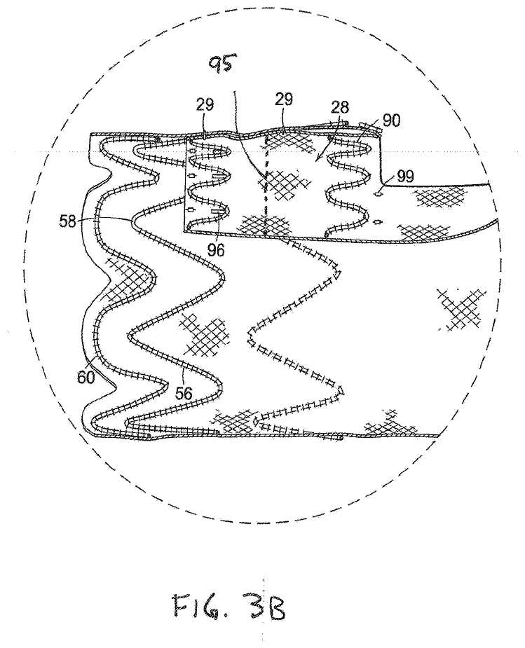

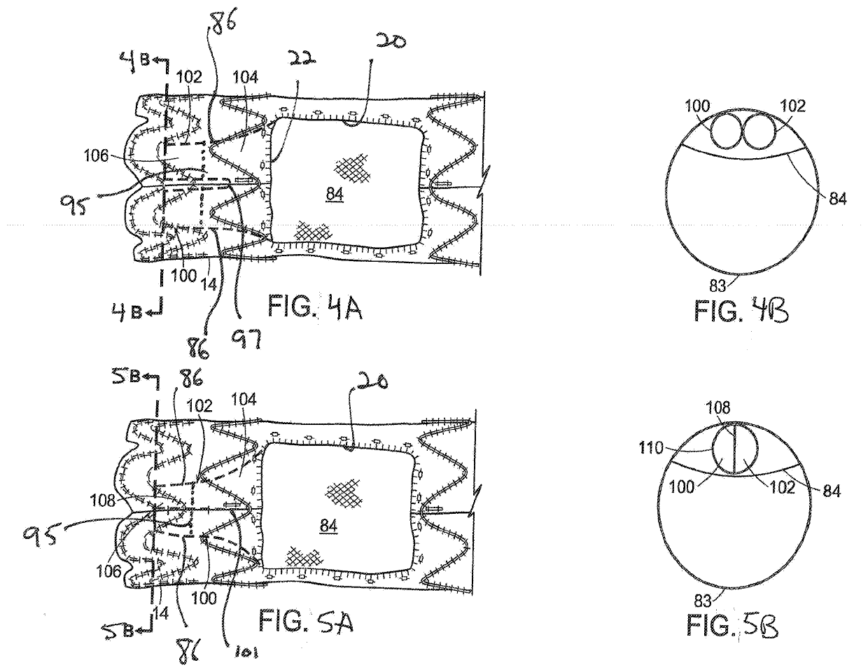

In one embodiment, shown in FIGS. 4A and 5A, the interface between tubular aortic component 12 and wall aperture 20, when viewed orthogonally to major longitudinal axis 24 of tubular aortic component 12 is a polygon, such as is shown in the referenced figures, a polygon having four sides. In various embodiments, the polygon can be a square, a rectangle, a parallelogram, or a rhombus (not shown).

In a specific embodiment, inferior portion 83 is on one side of tubular aortic component 12 opposite wall aperture 20 and is essentially parallel to major longitudinal axis 24 of tubular aortic component 12, shown in FIG. 2. Exposed apices 58 of clasping stent 56, when collapsed will cause at least partial collapse of proximal end 14 of tubular aortic component 12 at clasping stent 56. In one embodiment, distal end 32 of tunnel graft 28 has a diameter greater than that of proximal end 30 of tunnel graft 28, as can be seen in FIG. 4A. In another embodiment, proximal end 30 of tunnel graft 28 is between the most proximal edge of proximal end 14 of tubular aortic component 12 and proximal end 22 of wall aperture 20, as shown in FIGS. 3A, 4A, 5A, 6, and 7. As shown in FIG. 3B, tunnel graft 28 is secured to an interior wall of tubular aortic component 12 by a suitable means, such as by sutures 29.

As can be seen in FIGS. 4A and 5A, tunnel graft 28 includes open portion 84 at wall aperture 20 and tubular portions 86. Bifurcation point 97 marks the distal end of tubular portions 86 and the proximal end of open portion 84. Proximal end 22 of aperture 20 is distal to the proximal end of open portion 84. Open portion 84 extends proximally from aperture 22. In one embodiment, tunnel graft 28 includes stents 88, 90 at each of a proximal 92 and distal end 94 of tubular portion 86, as shown in FIGS. 3A, 3B, and 7. Preferably, stents 88, 90 at proximal end 92 and distal end 94 of tubular portion 86 includes proximal and distal apices connected by struts. Filters 95 are affixed to, such as by sewing and span internal lumens defined by tubular portions 86.

In another embodiment, shown in FIG. 6, filter 103 spans aperture 20 of tubular aortic component 12. Preferably, stent 88 at proximal end 92 of tubular portion 86 includes at least one barb 96 (FIGS. 3A and 3B). In another embodiment shown in FIG. 7, barbs 96 extend from distal apices of stent 98 of tubular portion 86. Optionally, tubular portion 86 further includes at least one stent 98 between stents 88,90 at proximal 92 and distal 94 ends, respectively, of tubular portion 86. Preferably, at least one of stents 98 between stents 88, 90 at proximal end 92 and distal end 94 includes at least one barb. Most preferably, stents of tubular portion 86 include nitinol. Filters 94 are affixed to, such as by sewing, and span internal lumens defined by tunnel grafts of FIG. 7 between at least two of 90, 92, and 98.

As can also be seen in FIGS. 3A and 3B, distal end 94 of tubular portion 86 is generally conical, whereby distal end 94 of tubular portion 86 essentially matches proximal end 92 of tunnel graft 28 at proximal end 22 of wall aperture 20, as a continuum or, optionally, at a seam, not shown. In one embodiment, a maximum diameter of proximal end of tunnel graft 28 is equal to or less than the diameter of distal end of tubular portion 94. Examples of suitable maximum diameters of proximal end 30 of tunnel graft 28 include, for example, diameters equal to or greater than a diameter selected from the group consisting of about 6 mm, about 8 mm, about 10 mm, about 12 mm or about 14 mm.

Preferably, tubular portion 86 has a major longitudinal axis that is parallel to major longitudinal axis 24 of tubular aortic component 12. Proximal end 92 of tubular portion 86 is distal to the most proximal edge of proximal end 14 of tubular aortic component 12. In one embodiment, not shown, proximal end 92 of tubular portion 86 is coterminous with the most proximal edge of proximal end 14 of tubular aortic component 12 or, alternatively, as shown in FIGS. 3A, 3B, 4A, 5A, 6, and 7, is distal to proximal end 14 of tubular aortic component 12.

In one embodiment, at least one radiopaque marker 99 is located at at least one of proximal end 92 of tunnel graft 28 and distal end 94 of tubular portion 86 of tunnel graft 28, as shown in FIGS. 3A and 3B. In still another embodiment, shown in FIGS. 3A, 3B, 6 and 7, proximal end 92 of tunnel graft 28 has a diameter in a range between about 5 mm and about 10 mm, or between about 5 mm and about 15 mm, or between about 8 mm and about 15 mm. Generally, tubular portion 86 has a length in a range of between about 20 mm and about 60 mm, or between about 20 mm and about 100 mm. Most commonly, tubular portion 86 has a length in a range between about 30 and 50 mm. Preferably, proximal end 92 of tunnel graft 28 is within at least about 5 mm, about 10 mm, and about 15 mm or about 20 mm of proximal end 14 of tubular aortic component 12.

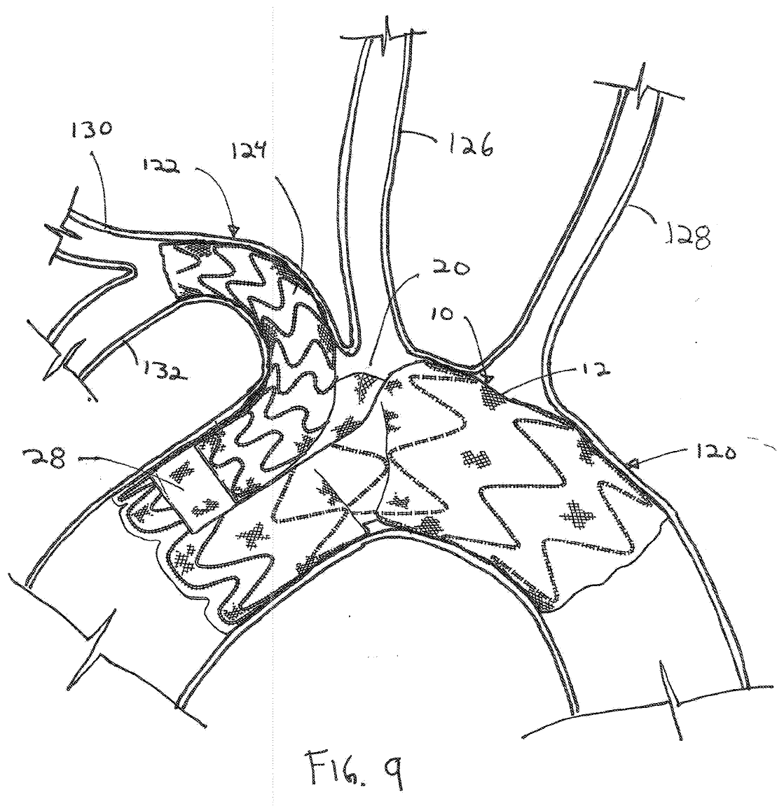

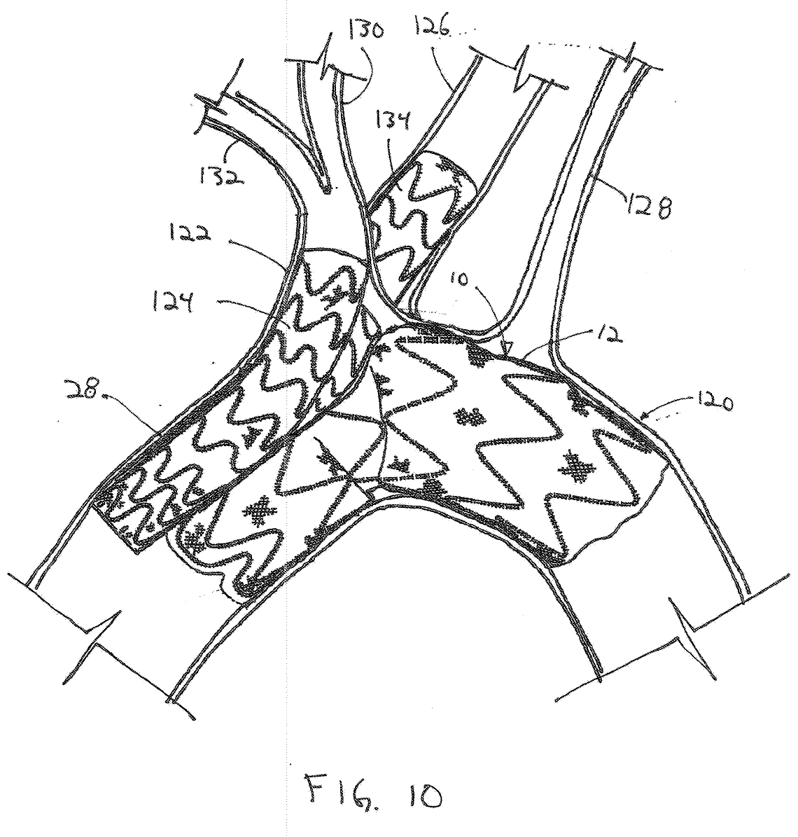

In an embodiment, represented in FIGS. 8 through 10, the method of the invention includes the step of implanting aortic graft assembly 10 of the invention at a surgical site, such as an aortic arch 120 of a patient, as can be seen in FIG. 8. During implantation of the aortic graft assembly 10 of the invention, emboli may form that, if allowed to escape the surgical site (e.g., the site of the aneurysm being treated with the aortic graft assembly), would pose a risk of stroke to the patient. Filter 95 sewn into and spanning at least one of 20 aperture of the tubular aortic component 12 of aortic graft assembly 10 and at least one tunnel graft of the aortic graft assembly of the invention sequester, or capture any such emboli, thereby preventing their escape from the surgical site. In one embodiment, after implantation, aperture 20 spans innominate artery (also referred to as “brachiocephalic artery”) 122 where it meets aortic arch 120.

Thereafter, at least one tubular branch component 124 is implanted in at least one of an innominate artery 122, as shown in FIG. 9, left common carotid artery 126, left subclavian artery 128, right common carotid artery 130, or right subclavian artery 132 of the patient and through wall aperture 20 into tunnel graft 28 within tubular aortic component 12, thereby rupturing the filter 95 spanning aperture 20 or tunnel graft 28. Rupture of filter 95 sequestering the emboli does not release the emboli back into the blood stream of the patient. Rather, the emboli remain sequestered, or captured, by the filter. In a preferred embodiment, the method of the invention includes the steps of implanting branch component 124 into innominate artery 122, and another tubular branch component 134 into the left common carotid artery 126.

Suitable systems, delivery devices and components of systems, stent grafts as described in U.S. application Ser. No. 11/699,700, filed on Jan. 30, 2007 (now abandoned); Ser. No. 11/828,653, filed on Jul. 26, 2007 (now abandoned); Ser. No. 12/137,592, filed on Jun. 12, 2008 (now abandoned); and Ser. No. 11/701,876, filed on Feb. 1, 2007 (now abandoned); and U.S. Pat. Nos. 7,763,063; 8,007,605; 8,062,345; 8,062,349; 8,070,790; 8,292,943 and U.S. Pat. Nos. 8,308,790, 8,740,963, 9,198,786, 9,320,631, 9,364,314, and 9,592,112, the relevant teachings of all of which are hereby incorporated by reference in their entirety, can be employed to deliver the aortic graft assembly of the invention by the method of the invention.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An aortic graft assembly, comprising:
a) a tubular aortic component that defines a tunnel lumen and includes a proximal end and a distal end connected by a wall of the tubular aortic component, the wall defining a wall aperture that is between the proximal and distal ends, the wall aperture having a proximal end and a distal end;
b) a tunnel graft connected to the wall of the tubular aortic component and extending from the wall aperture and within the tunnel lumen of the tubular aortic component toward the proximal end of the tubular aortic component, the tunnel graft having a proximal end and a distal end, and defining a tunnel graft lumen that extends between the distal end and the proximal end of the tunnel graft lumen, the distal end being at or proximal to the wall aperture of the tubular aortic component;
c) a proximal stent that supports the proximal end of the tubular aortic component;
d) a distal stent that supports the distal end of the tubular aortic component; and
e) at least one filter spanning at least one of the aperture and the tunnel graft.

2. The aortic graft assembly of claim 1, wherein the filter has a mesh size of between about 90 μm and about 100 μm.

3. The aortic graft assembly of claim 2 wherein the filter includes at least on of nylon, nitinol, and polyester.

4. The aortic graft assembly of claim 3, wherein the tunnel graft includes a first stent at a proximal end of the tunnel graft, and a second stent distal to the first stent.

5. The aortic graft assembly of claim 4, wherein the filter spans the tunnel graft between the first stent and the second stent.

6. The aortic graft assembly of claim 1, wherein the filter spans the aperture.

7. The aortic graft assembly of claim 1, wherein the tunnel graft is bifurcated, each tunnel graft having a proximal end and a distal end, and each tunnel graft further including first stent and a second stent.

8. The aortic graft assembly of claim 7, wherein the distal end of each tunnel graft lumen is proximal to the proximal end of the aperture.

9. The aortic graft assembly of claim 8, including at least two filters, and wherein at least one of the filters spans each tunnel graft lumen.

10. The aortic graft assembly of claim 9, where each of the tunnel graft lumens includes a first stent at the proximal end and a second stent at the distal end.

11. The aortic graft assembly of claim 10, wherein at least one of the filters is between the proximal and the first stent and the second stent of the respective tunnel graft.

12. The aortic graft assembly of claim 1, where the tunnel graft includes at least three stents between the proximal end and the distal end of the tunnel graft, and wherein at least one of the filters spans the tunnel graft between two of the stents of the tunnel graft.

13. The aortic graft assembly of claim 1, wherein the proximal end of the wall aperture includes an arch that lies in a first plane extending perpendicular to a major longitudinal axis of the tubular aortic component when viewed orthogonally to the major longitudinal axis.

14. The aortic graft assembly of claim 2, wherein the length of the proximal end of the wall aperture in the first plane is greater than the diameter of the tunnel graft lumen in a second plane extending orthogonally to the major longitudinal axis at a point proximal to the proximal end of the wall aperture.

15. A method for implanting a prosthesis, comprising the steps of:
a) delivering an aortic graft assembly that includes a tubular aortic component through an aorta to an aneurysm site of a patient, the tubular aortic component defining a tunnel lumen and having a proximal end and a distal end connected by a wall, the wall defining a wall aperture that is between the proximal end and the distal end, the wall aperture having a proximal end and a distal end, the aortic graft assembly further including a tunnel graft extending from the wall aperture and within the tunnel lumen of the tubular aortic component toward the proximal end of the tubular aortic component, wherein at least one filter spans at least one of the aperture and the tunnel graft;
b) aligning the wall aperture over at least one vessel ostium at the aneurysm site of the patient; and
c) retracting the outer control tube, thereby releasing the tubular aortic component from the distal and proximal clasps, thereby deploying the tubular aortic component at the aneurysm site in the patient.

16. The method of claim 15, further including the step of directing a branch stent graft from an aortic branch through the aperture and at least partially through the tunnel graft, thereby rupturing the filter.

17. The method of claim 16, wherein the proximal end of the wall aperture includes an arch that lies in a first plane extending perpendicular to a major longitudinal axis of the tubular aortic component when viewed orthogonally to the major longitudinal axis, the tubular aortic component being radially and releasably constrained by a distal clasp at a distal end of an outer control tube of a delivery device, and releasably attached by a retention component to a proximal clasp at the outer control tube proximal to the proximal clasp, the tubular aortic component further supported by a control catheter of the delivery device extending within the outer control tube.

18. The method of claim 17, wherein the length of the proximal end of the wall aperture in the first plane is greater than the diameter of the tunnel graft lumen in a second plane extending orthogonally to the major longitudinal axis at a point proximal to the proximal end of the wall aperture.
